SEQUENCE LISTING (13 sequences)

SEQ ID NO: 1 - DNA, 20 bp, Artificial Sequence
  Left primer for TIMELESS gene
ttgcagaactggaggtgttg

SEQ ID NO: 2 - DNA, 20 bp, Artificial Sequence
  Right primer for TIMELESS gene
aggttgtgaaggcctttgtg

SEQ ID NO: 3 - DNA, 20 bp, Artificial Sequence
  Left primer for CDC6 gene
gctgttgaacttcccacctt

SEQ ID NO: 4 - DNA, 20 bp, Artificial Sequence
  Right primer for CDC6 gene
tctcctgcaaacatccagtg

SEQ ID NO: 5 - DNA, 20 bp, Artificial Sequence
  Left primer for DNMT3B gene
agatcaagctcgcgactctc

SEQ ID NO: 6 - DNA, 20 bp, Artificial Sequence
  Right primer for DNMT3B gene
gacagctgggctttctgaac

SEQ ID NO: 7 - DNA, 20 bp, Artificial Sequence
  Left primer for DNMT3A gene
ggacaagaatgccaccaaag

SEQ ID NO: 8 - DNA, 20 bp, Artificial Sequence
  Right primer for DNMT3A gene
ccactgagaatttgccgtct

SEQ ID NO: 9 - DNA, 20 bp, Artificial Sequence
  Left primer for MYBL2 gene
cacctggaggaggacttgaa

SEQ ID NO: 10 - DNA, 20 bp, Artificial Sequence
  Right primer for MYBL2 gene
ccacaatgtcaagagccaga

SEQ ID NO: 11 - DNA, 20 bp, Artificial Sequence
  Left primer for CBX1 gene
ggagcggattattggagcta

SEQ ID NO: 12 - DNA, 20 bp, Artificial Sequence
  Right primer for CBX1 gene
atcctccgaggggtaggaat

SEQ ID NO: 13 - DNA, 22 bp, unknown
  miR-29a oligonucleotide sequence
tagcaccatctgaaatcggtta

(19) United States
(12) Patent Application Publication
(10) Pub. No.: US 2012/0251619 A1
(43) Pub. Date: Oct. 4, 2012

(54) MicroRNA-29a,b,c as a Tumor Suppressor and Sensitizing Agent for Chemotherapy

(76) Inventor: Preethi H. Gunaratne, Houston, TX (US)

(21) Appl. No.: 13/437,128

(22) Filed: Apr. 2, 2012

Related U.S. Application Data
(60) Provisional application No. 61/516,240, filed on Mar. 31, 2011.

(51) Int. Cl. A61K 31/713; A61K 9/127; A61N 5/00; C40B 30/04; C12Q 1/68; A61K 33/24; A61P 35/00

(52) U.S. Cl. 424/450; 514/44 A; 424/649; 506/9; 435/6.12; 600/1

(57) ABSTRACT

The present invention provides a method of improving a therapeutic response to a cancer treatment, in a subject, the method comprising administering an effective amount of an agent that enhances the expression of microRNA 29 or an agent that mimics the effects of microRNA 29. Further provided is a method of treating a cancer in a subject in need of such treatment comprising the step of administering an effective amount of a microRNA 29 or an agent that enhances the expression of microRNA 29.

### CROSS-REFERENCES TO RELATED APPLICATIONS

This non-provisional application claims benefit of provisional application U.S. Ser. No. 61/516,240, filed Mar. 31, 2011, now abandoned, the entirety of which is hereby incorporated by reference.

### BACKGROUND OF THE INVENTION

1. Field of the Invention

The present invention relates generally to the fields of microRNA molecular biology and cancer. More specifically, the invention relates to the use of microRNA 29a,b,c as tumor suppressors able to significantly suppress cell proliferation, increase apoptosis, suppress tumor growth and increase sensitivity of chemotherapeutic drugs.

2. Description of the Related Art

RNA interference refers to the process of sequence-specific post-transcriptional gene silencing in animals mediated by short interfering RNAs (siRNAs) (Fire et al. (1998) Nature 391:806-810). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al. (1999) Trends Genet. 15:358-363). Such protection from foreign gene expression may have evolved in response to the production of double-stranded RNAs (dsRNAs) derived from viral infection or from the random integration of transposon elements into a host genome via a cellular response that specifically destroys homologous single-stranded RNA of viral genomic RNA.

The presence of dsRNA in cells triggers the RNAi response through a mechanism that has yet to be fully characterized. The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Bernstein et al. (2001) Nature 409:363-366). Short interfering RNAs derived from dicer activity are typically about 21 to about 23 nucleotides in length and comprise about 19 base pair duplexes (Elbashir et al. (2001) Genes Dev 15:188-200). Dicer has also been implicated in the excision of 21- and 22-nucleotide small temporal RNAs (stRNAs) from precursor RNA of conserved structure that are implicated in translational control (Hutvagner et al. (2001) Science 293:834-838). The RNAi response also features an endonuclease complex, commonly referred to as an RNA-induced silencing complex (RISC), which mediates cleavage of single-stranded RNA having sequence complementarity to the antisense strand of the siRNA duplex. Cleavage of the target RNA takes place in the middle of the region complementary to the antisense strand of the siRNA duplex (Elbashir et al. (2001) Genes Dev 15:188-200). In addition, RNA interference can also involve small RNA (e.g., microRNA, or miRNA) mediated gene silencing, presumably through cellular mechanisms that regulate chromatin structure and thereby prevent transcription of target gene sequences (see, e.g., Allshire, Science 297:1818-1819 2002; Volpe et al. (2002) Science 297:1833-1837; Jenuwein (2002) Science 297:2215-2218; Hall et al. (2002) Science 297:2232-2237).

As such, miRNA molecules of the invention can be used to mediate gene silencing via interaction with RNA transcripts or alternately by interaction with particular gene sequences, wherein such interaction results in gene silencing either at the transcriptional or post-transcriptional level. RNAi has been studied in a variety of systems. Fire et al. ((1998) Nature 391:806-811) were the first to observe RNAi in C. elegans. Wianny and Goetz ((1999) Nature Cell Biol 2:70) describe RNAi mediated by dsRNA in mouse embryos. Hammond et al. ((2000) Nature 404:293-296) describe RNAi in Drosophila cells transfected with dsRNA. Elbashir et al. ((2001) Nature 411:494-498) describe RNAi induced by introduction of duplexes of synthetic 21-nucleotide RNAs in cultured mammalian cells including human embryonic kidney and HeLa cells. Small RNAs play an important role in controlling gene expression.

Regulation of many developmental processes is controlled by small RNAs. It is now possible to engineer changes in gene expression of plant genes by using transgenic constructs which produce small RNAs in the plant. Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited. It is thought that sequence complementarity between small RNAs and their RNA targets helps to determine which mechanism, RNA cleavage or translational inhibition, is employed. It is believed that siRNAs, which are perfectly complementary with their targets, work by RNA cleavage. Some miRNAs have perfect or near-perfect complementarity with their targets, and RNA cleavage has been demonstrated for at least a few of these miRNAs. Other miRNAs have several mismatches with their targets, and apparently inhibit their targets at the translational level.

Again, without being held to a particular theory on the mechanism of action, a general rule is emerging that perfect or near-perfect complementarity favors RNA cleavage, especially within the first ten nucleotides (counting from the 5′ end of the miRNA), whereas translational inhibition is favored when the miRNA/target duplex contains many mismatches. The apparent exception to this is microRNA 172 (miR172) in plants. One of the targets of miR172 is APETALA2 (AP2), and although miR172 shares near-perfect complementarity with AP2 it appears to cause translational inhibition of AP2 rather than RNA cleavage. MicroRNAs (miRNAs) are noncoding RNAs of about 19 to about 24 nucleotides (nt) in length that have been identified in both animals and plants (Lagos-Quintana et al. (2001) Science 294:853-858, Lagos-Quintana et al. (2002) Curr Biol 12:735-739; Lau et al. (2002) Science 294:858-862; Lee and Ambros (2001) Science 294:862-864; Llave et al. (2002) Plant Cell 14:1605-1619; Mourelatos et al. (2002) Genes Dev 16:720-728; Park et al. (2002) Curr Biol 12:1484-1495; Reinhart et al. (2002) Genes Dev 16:1616-1626). They are processed from longer precursor transcripts that range in size from approximately 70 to 200 nt, and these precursor transcripts have the ability to form stable hairpin structures.

In animals, the enzyme involved in processing miRNA precursors is called Dicer, an RNAse III-like protein (Grishok et al. (2001) Cell 106:23-34; Hutvagner et al. (2001) Science 293:834-838; Ketting et al. (2001) Genes Dev 15:2654-2659). Plants also have a Dicer-like enzyme, DCL1 (previously named CARPEL FACTORY/SHORT INTEGUMENTS1/SUSPENSOR1), and recent evidence indicates that it, like Dicer, is involved in processing the hairpin precursors to generate mature miRNAs (Park et al. (2002) Curr Biol 12:1484-1495; Reinhart et al. (2002) Genes Dev 16:1616-1626). Furthermore, it is becoming clear from recent work that at least some miRNA hairpin precursors originate as longer polyadenylated transcripts, and several different miRNAs and associated hairpins can be present in a single transcript (Lagos-Quintana et al. (2001) Science 294:853-858; Lee et al. (2002) EMBO J. 21:4663-4670). Recent work has also examined the selection of the miRNA strand from the dsRNA product arising from processing of the hairpin by DICER (Schwartz et al. (2003) Cell 115:199-208). It appears that the stability (i.e. G:C vs. A:U content, and/or mismatches) of the two ends of the processed dsRNA affects the strand selection, with the low stability end being easier to unwind by a helicase activity. The 5′ end strand at the low stability end is incorporated into the RISC complex, while the other strand is degraded. In animals, there is direct evidence indicating a role for specific miRNAs in development.

The lin-4 and let-7 miRNAs in C. elegans have been found to control temporal development, based on the phenotypes generated when the genes producing the lin-4 and let-7 miRNAs are mutated (Lee et al. (1993) Cell 75:843-854; Reinhart et al. (2000) Nature 403-901-906). In addition, both miRNAs display a temporal expression pattern consistent with their roles in developmental timing. Other animal miRNAs display developmentally regulated patterns of expression, both temporal and tissue-specific (Lagos-Quintana et al. (2001) Science 294:853-853, Lagos-Quintana et al. (2002) Curr Biol 12:735-739; Lau et al. (2001) Science 294:858-862; Lee and Ambros (2001) Science 294:862-864), leading to the hypothesis that miRNAs may, in many cases, be involved in the regulation of important developmental processes. Likewise, in plants, the differential expression patterns of many miRNAs suggests a role in development (Llave et al. (2002) Plant Cell 14:1605-1619; Park et al. (2002) Curr Biol 12:1484-1495; Reinhart et al. (2002) Genes Dev 16:1616-1626), which has now been shown (e.g., Guo et al. (2005) Plant Cell 17:1376-1386).

MicroRNAs appear to regulate target genes by binding to complementary sequences located in the transcripts produced by these genes. In the case of 11n-4 and let-7, the target sites are located in the 3′ UTRs of the target mRNAs (Lee et al. (1993) Cell 75:843-854; Wightman et al. (1993) Cell 75:855-862; Reinhart et al. (2000) Nature 403:901-906; Slack et al. (2000) Mol Cell 5:659-669), and there are several mismatches between the lin-4 and let-7 miRNAs and their target sites. Binding of the lin-4 or let-7 miRNA appears to cause downregulation of steady-state levels of the protein encoded by the target mRNA without affecting the transcript itself (Olsen and Ambros (1999) Dev Biol 216:671-680). On the other hand, recent evidence suggests that miRNAs can, in some cases, cause specific RNA cleavage of the target transcript within the target site, and this cleavage step appears to require 100% complementarity between the miRNA and the target transcript (Hutvagner and Zamore (2002) Science 297:2056-2060; Llave et al. (2002) Plant Cell 14:1605-1619), especially within the first ten nucleotides (counting from the 5′ end of the miRNA).

It seems likely that miRNAs can enter at least two pathways of target gene regulation. Protein downregulation when target complementarity is <100%, and RNA cleavage when target complementarity is 100%. MicroRNAs entering the RNA cleavage pathway are analogous to the 21-25 nt short interfering RNAs (siRNAs) generated during RNA interference (RNAi) in animals and posttranscriptional gene silencing (PTGS) in plants (Hamilton and Baulcombe (1999) Science 286:950-952; Hammond et al., (2000) Nature 404:293-296; Zamore et al., (2000) Cell 31:25-33; Elbashir et al., (2001) Nature 411:494-498), and likely are incorporated into an RNA-induced silencing complex (RISC) that is similar or identical to that seen for RNAi.

Identifying the targets of miRNAs with bioinformatics has not been successful in animals, and this is probably due to the fact that animal miRNAs have a low degree of complementarity with their targets. On the other hand, bioinformatic approaches have been successfully used to predict targets for plant miRNAs (Llave et al. (2002) Plant Cell 14:1605-1619; Park et al. (2002) Curr Biol 12:1484-1495; Rhoades et al. (2002) Cell 110:513-520), and thus it appears that plant miRNAs have higher overall complementarity with their putative targets than do animal miRNAs. Most of these predicted target transcripts of plant miRNAs encode members of transcription factor families implicated in plant developmental patterning or cell differentiation. Nonetheless, biological function has not been directly demonstrated for any plant miRNA. Although Llave et al. ((2002) Science 297:2053-2056) have shown that a transcript for a SCARECROW-like transcription factor is a target of the Arabidopsis miRNA mir171, these studies were performed in a heterologous species and no plant phenotype associated with mir171 was reported.

General categories of sequences of interest for the invention described include, for example, those genes involved in regulating oncogenic processes that are responsible for the initiation, progression or maintenance of increased cell proliferation and/or decreased cell death that are direct or indirect targets of tumor suppressor microRNAs. Target sequences further include coding regions and non-coding regions such as promoters, enhancers, terminators, introns and the like, which may be modified in order to alter the expression of a gene of interest. For example, an intron sequence can be added to the 5′ region to increase the amount of mature message that accumulates (see for example Buchman and Berg (1988) Mol Cell Biol 8:4395-4405); and Callis et al. (1987) Genes Dev 1:1183-1200). This current invention is about microRNAs are small-22 nucleotide non-coding RNAs that can bind protein coding mRNAs through complimentary base pairing to mediate mRNA decay or translational repression. Because a single microRNA can bind and silence hundreds of genes across diverse signaling pathways they can be developed as powerful therapeutic agents to silence entire disease networks.

The prior art is deficient in the use of the miR-29a, miR-29b, miR-29c microRNA family to, inter alia, significantly enhance sensitivity to chemotherapeutic drug as well as provide an alternative or complement to small molecule inhibitor treatment for ovarian and other cancers.

### SUMMARY OF THE INVENTION

The present invention is directed to the use of the miR-29a, miR-29b, miR-29c microRNA family to, inter alia, significantly enhance sensitivity to chemotherapeutic drug as well as provide an alternative or complement to small molecule inhibitor treatment for ovarian and other cancers when presented in the form of pri-miRNA, pre-miRNA, mature miRNA or fragments of variants thereof that retain the biological activity of the mature miRNA and DNA encoding a pri-miRNA, pre-miRNA, mature miRNA, fragments or variants thereof, or regulatory elements of the miRNA.

Thus, in one embodiment of the present invention, there is provided a method of providing a prognosis for ovarian cancer in a subject, comprising the steps of: obtaining a biological sample from said subject; and testing said biological sample to determine whether or not microRNA 29 is under-expressed in said sample, relative to the expression of microRNA 29 in a control sample, whereby the under-expression of microRNA 29 in said biological sample indicates that a tumor in said subject is resistant to a chemotherapy.

In another embodiment of the present invention, there is provided a method of improving a therapeutic response to a cancer treatment in a subject, the method comprising administering an effective amount of a microRNA 29 or an agent that mimic the effects or enhance expression of microRNA 29. Representative microRNA 29 compounds include microRNA 29a, microRNA 29b or microRNA 29c. Representative agents that mimic the effects or enhance expression of microRNA 29 include but are not limited to double-stranded miRNA 29 mimics and oligonucleotide based pre-microRNA 29 drug. Representative cancers include but are not limited to lung cancer, pancreatic cancer, skin cancer, hematological neoplasms, breast cancer, brain cancer, colon cancer, follicular lymphoma, bladder cancer, cervical cancer, endometrial cancer, esophageal cancer, gastric cancer, head and neck cancer, multiple myeloma, liver cancer, lymphomas, oral cancer, osteosarcomas, ovarian cancer, uterine leiomyosarcoma, uterine leiomyomas, endometriomas, endometriosis, uterine papillary serous carcinomas, prostate cancer, testicular cancer and thyroid cancer. In a preferred embodiment the cancer is epithelial ovarian cancer. Representative therapeutic response include but are not limited to treating with radiation, carboplatin, cisplatin, paclitaxel, an alkylating agent, an antimetabolite, an antitumor antibiotic, and a DNA topoisomerase inhibitor.

In yet another embodiment of the present invention, there is provided a kit for determining a chemotherapy response in a patient with a cancer, said kit comprising: a) a oligonucleotide complementary to microRNA 29; and b) optionally, reagents for the formation of the hybridization between said oligonucleotide and said microRNA 29. In one aspect, the microRNA 29 may be detectably labeled. A person with ordinary skill in this art would recognize that, in this kit, the microRNA 29 could attached to a solid surface. For example, the microRNA 29 could be a member of a nucleic acid array. A representative example of a nucleic acid array is a micro-array.

In yet another embodiment of the present invention, there is provided a pharmaceutical composition for improving a tumor response to chemotherapy, said composition comprising an effective amount of microRNA 29 or an agent that enhances the expression of microRNA 29 or mimics the actions of microRNA 29.

In yet another embodiment of the present invention, there is provided a method of treating a cancer in a subject in need of such treatment comprising the step of administering an effective amount of a microRNA 29 or an agent that enhances the expression of microRNA 29 or mimics the actions of microRNA 29. In this method, the microRNA 29 may be microRNA 29a, microRNA 29b or microRNA 29c. Representative examples of an agent that enhances the expression of microRNA 29 or mimics the actions of microRNA 29 include double-stranded miRNA mimics and oligonucleotide based pre-microRNA 29 drugs. Representative examples of cancer which may be treated using this method include but are not limited to lung cancer, pancreatic cancer, skin cancer, hematological neoplasms, breast cancer, brain cancer, colon cancer, follicular lymphoma, bladder cancer, cervical cancer, endometrial cancer, esophageal cancer, gastric cancer, head and neck cancer, multiple myeloma, liver cancer, lymphomas, oral cancer, osteosarcomas, ovarian cancer, uterine leiomyosarcoma, uterine leiomyomas, endometriomas, endometriosis, uterine papillary serous carcinomas, prostate cancer, testicular cancer and thyroid cancer. In a preferred aspect, the cancer is epithelial ovarian cancer.

In a further embodiment, this method may further comprising treating said subject with radiation, carboplatin, cisplatin, paclitaxel, an alkylating agent, an antimetabolite, an antitumor antibiotic and a DNA topoisomerase inhibitor. A person having ordinary skill in this art would readily recognize that the microRNA 29 or agent that enhances the expression of microRNA 29 or mimics the actions of microRNA 29 may be administered as a nucleic acid construct encoding an artificial miRNA presented as a double-stranded RNA, a precursor hairpin, a primary miRNA in single straded RNA form or encoded in a DNA vector delivered in a suitable pharmaceutical carrier. Representative examples of pharmaceutical carrier which may be used in this method include but are not limited to a virus, a liposome, and a polymer. The method of claim 15, wherein said microRNA 29 is administered as a nanoparticle, a liposome, a vector or a polymer. Representative examples of vector which may be used in this method include but are not limited to a plasmid, a cosmid, a phagemid and a virus.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1: Gene transcripts with miRNA 7mer in the 3′-UTR tend to be anti-correlated with expression of the corresponding miRNA. The scatter plot shows mean correlation versus significance of enrichment for predicted target interactions (enrichment expressed as a Fisher's exact z-score), when separately considering the following potential interactions: 7mer seed sequence in 3′-UTR (black dotted line), 7mer seed sequence in 5′-UTR (black dashed line), 7mer seed sequence in coding sequence region (.cds,. gray dotted line), miRanda prediction (black solid line), TargetScan prediction (gray solid line). Plot uses bins of 10000 miRNA:mRNA pairs (total number of pairs represented: 191 miRNAs X 8547 genes). Fisher's exact z-score of +/−2.57 corresponds to significant enrichment (nominal P<0.01) for predicted targets within miRNA:mRNA pairs.

FIG. 2: Correlation of gene expression with miR-29a expression. Predicted miR-29a targets are indicated.

FIG. 3: Top eight words (of all 5, 6 and 7mers) enriched in 3′-UTRs of mRNAs anti-correlated with miR-29a expression (FDR<1⁻⁶).

FIG. 4: QPCR analysis showing relative quantity of selected miR-29a anti-correlated gene targets after miR29a overexpression in HEYA8 ovarian cancer cells (SCR, scrambled control; WT, untreated; two-sided t-test P<0.05, mir-29a vs SCR and miR-29a vs WT, each comparison, except for SAE1).

FIGS. 5A-5B: MTS assays demonstrating the effect of miR-29a overexpression on proliferation of HEYA8 (FIG. 5A) and OVCAR-8 cells (FIG. 5B) (Lipo, lipofectamine-treated alone, no miRNA; two-sided t-test P<0.001, miR-29a vs each of three control groups at both 48 h and 72 h). For the in vitro evaluation of miR-29a on cell proliferation of ovarian cancer cells, microRNA mimics of miR-29a were transiently transfected into the p53-deficient ovarian cancer cell line OVCAR8 and the p53-wild type HEYA8 and the impact of this microRNA on cell proliferation was measured by increase in absorbance from the MTT assay. When compared with the parental strains in each case as well as these cell lines transiently transfected with a scrambled control miR-29a is able to very significantly suppress cell proliferation in p53-wild type HEYA8 and moderately suppress cell proliferation of the p53-deficient OVCAR8.

FIGS. 6A-6B: Effect of miR-29a (72 h) on proliferation, under a range of concentrations of cisplatin treatment (at 48 h) of HEYA8 (FIG. 6A) and OVCAR-8 cells (FIG. 6B). Error bars are standard error. Proliferation curves of the parental HEYA8 strain treated with a scrambled control sequence and with microRNA mimics for miR-29a are shown. The data suggests that miR-29a is able to suppress the proliferation of HEYA8 significantly more effectively than the scrambled control at the same dosage of cisplatin.

### DESCRIPTION OF THE PREFERRED EMBODIMENTS

As used herein, the term “a” or “an”, when used in conjunction with the term “comprising” in the claims and/or the specification, may refer to “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

As used herein, the term “or” in the claims refers to “and/or” unless explicitly indicated to refer to alternatives only or the alternatives are mutually exclusive, although the disclosure supports a definition that refers to only alternatives and “and/or.”

The present invention relates to the design, synthesis, construction, composition, characterization and use of a novel therapeutic agent such as nucleic acids (microRNAs) and methods useful in treating cancer. More specifically, the invention discloses that artificial microRNA 29a,b,c is a potent tumor suppressor able to significantly suppress cell proliferation, increase apoptosis, suppress tumor growth and increase sensitivity of chemotherapeutic drugs when presented in the form of pri-miRNA, pre-miRNA, mature miRNA or fragments of variants thereof that retain the biological activity of the mature miRNA and DNA encoding a pri-miRNA, pre-miRNA, mature miRNA, fragments or variants thereof, or regulatory elements of the miRNA.

A preferred embodiment of the present invention discloses that that miR-29a significantly decreases the proliferation of both p53-deficient OVCAR8 and p53-wild type HEYA8. This is common characteristic of tumor suppressor genes and microRNAs. From this work, a person having ordinary skill in this art could readily conclude that miR-29a along with its family members mir-29b and miR-29c are strong suppressors of ovarian and other cancers.

Another preferred embodiment of the present invention discloses that miR-29a significantly increases the sensitivity to cisplatin (which is commonly used to treat ovarian and other cancers). miR-29a treated HEYA8 cells proliferate at rates substantially lower than HEYA8 cells treated with a scrambled control or the parental HEYA8 cell line. From these data one may readily conclude that miR-29a and its family members miR-29b and miR-29c would significantly increase the sensitivity of tumors to chemotherapy in ovarian and other cancers.

Another preferred embodiment of this invention teaches that patients that are able to respond to current doses of chemotherapy can be treated with much lower doses of chemotherapy when presented with miR-29a,b,c. Also, patients that do not respond to chemotherapy, or patients that respond but relapse, can be treated with regular doses of chemotherapy in presence of miR-29a,b,c. In addition since miR-29a is highly effective at suppressing the proliferation of p53-wild type ovarian cancer cells it is likely to be effective in treating low grade tumors as well.

One preferred embodiment of the invention discloses the use of a nucleic acid construct encoding an artificial miRNA presented as a double-stranded RNA or precursor hairpin or a primary miRNA in the single straded RNA form or encoded in a DNA vector delivered in a suitable pharmaceutical carrier, to be used for inhibiting the expression of all oncogenes and regulators of oncogenes containing a miR-29a,b,c complementary site (LCS). The pharmaceutical carrier includes, but is not limited to, a virus, a liposome, or a polymer, and any combination thereof.

Another preferred embodiment of the present invention discloses the composition, methods and use of a nucleic acid construct encoding an artificial miRNA presented as a double-stranded RNA or precursor a hairpin or a primary miRNA in the single stranded RNA form or encoded in a DNA vector delivered in a suitable pharmaceutical carrier, to be used for inhibiting the expression of all oncogenes and regulators of oncogenes containing a miR-29a,b,c complementary site (LCS), wherein the miR-29a,b,c is delivered in multiple ways, to include but not limited to, as a mature miRNA by itself, or as a gene is encoded by a nucleic acid, or as a precursor hairpin by itself or conjugated to nanoparticles of metal or liposomal origin, or conjugated to nanoparticles of metal or liposomal origin, or as a primary miRNA by itself or conjugated to nanoparticles of metal or liposomal origin or delivered on a virus, or as a liposome, or as a polymer, or as a gene that is encoded by a nucleic acid and such nucleic acid is located on a vector, or as a gene is encoded by a nucleic acid, or as a precursor hairpin by itself or conjugated to nanoparticles of metal or liposomal origin.

Another preferred embodiment of the present invention discloses that such nucleic acid is located on a vector selected from the group consisting of a plasmid, cosmid, phagemid, virus, and other vehicles derived from viral or bacterial sources, or is located on a vector that may further comprises one or more in vivo expression elements selected from the group consisting of a promoter, enhancer, and combinations thereof.

Another preferred embodiment of the present invention relates to the use of miR-29a,b,c where miRNA is administered to, or expression is increased in the cells of, a patient for treatment or prevention of cancer, including but not limited to lung cancer, pancreatic cancer, skin cancer, hematological neoplasms, breast cancer, brain cancer, colon cancer, follicular lymphoma, bladder cancer, cervical cancer, endometrial cancer, esophageal cancer, gastric cancer, head and neck cancer, multiple myeloma, liver cancer, lymphomas, oral cancer, osteosarcomas, ovarian cancer, uterine leiomyosarcoma, uterine leiomyomas, endometriomas, endometriosis, uterine papillary serous carcinomas, prostate cancer, testicular cancer, and/or thyroid cancer. Another preferred embodiment of the present invention relates to the use of miR-29a,b,c where miRNA is administered to, or expression is increased in the cells of, a patient for treatment or prevention of cancer and wherein the patient is undergoing one or more cancer therapies selected from the group consisting of surgery, chemotherapy, radiotherapy, thermotherapy, immunotherapy, hormone therapy and laser therapy. Another embodiment of the present invention discloses a method for determining the sensitivity of a cancer to a miR-29a,b,c miRNA delivered on a suitable pharmaceutical carrier to bind to an mRNA encoded by an oncogene containing one or several miR-29a,b,c complementary site (LCS) in a cancerous or transformed cell or an organism with a cancerous or transformed cell; and determining if the cancerous or transformed cell growth or viability is inhibited or if expression of the oncogene is inhibited. While the invention described here specifically relates to the design and construction of a novel therapeutic agents such as nucleic acids (microRNAs) to treat cancer, one of ordinary skills in the art, with the benefit of this disclosure, it is possible to extend the proposed microRNAs to be used in many kind of cancer treatment, and would recognize the extension of the approach to other treatment protocols.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Molecular Profiling Datasets

The set of 487 tumors analyzed were from the original TCGA set of 489 (samples TCGA-041536 and TCGA-61-1911 did not have quality miRNA data at the time of this study). The miRNA array normalization steps are as follows. The gMeanSignal from raw array files (.level 1.) were quantile normalized and log transformed, removing duplicate samples and control probes (.level 2.). Multiple median centering steps set the median of every batch to the median of all batches: in brief, within each batch, the median for each miRNA was first subtracted, then calculated the across batch median and added it back to all samples within that batch; the resulting data were collapsed to miRNA levels (.level 3.). The level 3 miRNA data are available at the TCGA Data Portal. For gene expression analysis, the previously described .unified. dataset was used.

The definition and validation of a prognostic miRNA signature was carried out essentially as described for the previously-defined prognostic mRNA (gene) signature [The_Cancer_Genome_Atlas_Research_Network (2011) Integrated genomic analyses of ovarian carcinoma. Nature 474: 609-615], using the previously-defined training and validation subsets with expression values normalized within each subset to standard deviations from the median. Given the miRNA signature from the training dataset, the prognostic t-score was defined for each validation profile as the two-sided t-statistic comparing, within each tumor profile, the average of the poor prognosis miRNAs with the average of the good prognosis miRNAs.

The time course MTS assay experiments were run three times (separate days), each with a different set of biological quadruplicates (n=12 per group); within each experiment run, the viability measures within each time point were centered on the mean of the WT group for the first run. For cisplatin treatment, cells were transfected as described, and media was replaced after 24 hrs with media containing cisplatin (Sigma) (0-7.5 μg/mL). Viability was assayed 72 hrs post-transfection. Experiments were run three times, each with a different biological replicate (n=3 per group). For each run, viability measures within each concentration point were centered on the mean of values for the first run.

### Example 2

### Quantitative Real-Time PCR (QPCR)

Total RNA (60 ng) was reverse transcribed in a 40 μl reaction using the TaqMan® MicroRNA Reverse Transcription Kit (ABI). Custom primer sequences are shown in Table 1. QPCR was performed on a StepOne Real-Time PCR System (ABI) using Power-SYBR Green PCR Master Mix (ABI) in a 20 μl reaction and human ribosomal RNA 18s as an endogenous control (which was itself not miR-29a-regulated, data not shown). The QPCR experiments were run four times (separate days), each with independent biological samples (n=4 per group); within each experiment run, relative expression values were normalized to standard deviations from the mean.

TABLE 1

| Target gene: | Primer sequence | | Sequence |
|---|---|---|---|
| TIMELESS | LEFT | 5′-TTGCAGAACTGGAGGTGTTG-3′ | SEQ ID NO: 1 |
| TIMELESS | RIGHT | 5′-AGGTTGTGAAGGCCTTTGTG | SEQ ID NO: 2 |
| CDC6 | LEFT | 5′-GCTGTTGAACTTCCCACCTT-3′ | SEQ ID NO: 3 |
| CDC6 | RIGHT | 5′-TCTCCTGCAAACATCCAGTG-3′ | SEQ ID NO: 4 |
| DNMT3B | LEFT | 5′-AGATCAAGCTCGCGACTCTC-3′ | SEQ ID NO: 5 |
| DNMT3B | RIGHT | 5′-GACAGCTGGGCTTTCTGAAC-3′ | SEQ ID NO: 6 |
| DNMT3A | LEFT | 5′-GGACAAGAATGCCACCAAAG-3′ | SEQ ID NO: 7 |
| DNMT3A | RIGHT | 5′-CCACTGAGAATTTGCCGTCT-3′ | SEQ ID NO: 8 |
| MYBL2 | LEFT | 5′-CACCTGGAGGAGGACTTGAA-3′ | SEQ ID NO: 9 |
| MYBL2 | RIGHT | 5′-CCACAATGTCAAGAGCCAGA-3′ | SEQ ID NO: 10 |
| CBX1 | LEFT | 5′-GGAGCGGATTATTGGAGCTA-3′ | SEQ ID NO: 11 |
| CBX1 | RIGHT | 5′-ATCCTCCGAGGGGTAGGAAT-3′ | SEQ ID NO: 12 |

### Example 3

### Results

MiRNAs are influenced by both copy number alteration and genomic location. The TCGA ovarian cancer datasets were examined, representing 487 tumors profiled for miRNA expression, for patterns of correlation between the miRNAs and other molecular features. To begin with, it was considered whether miRNAs with expression levels frequently altered by changes in DNA copy number may reveal a subset of miRNAs under clonal selection in the tumors. Such miRNAs would be of interest as candidate oncomiRs or tumor suppressive miRs. miRNAs were therefore systematically analyzed for both loss and gain of DNA copy number associated with a concordant change in mature miRNA expression level. This analysis revealed several miRNAs in focally amplified and deleted genomic regions. In particular, let-7b was the most frequently deleted miRNA having both recurrent hemizygous genomic loss (86% of samples) and homozygous deletion (7.2%). Four members of the miR-30 family were among the most frequently amplified miRNAs. Interestingly, these members were encoded at two different focally amplified loci (8q24 and 1p34) and all four miRNAs showed strong concordant change in mature miRNA expression.

Moreover, miRNAs were frequently coexpressed with neighboring miRNAs. Previously, when examining miRNA expression profiles in a small dataset of 24 normal human tissues, Baskerville and Bartel found evidence that proximal pairs of miRNAs are generally coexpressed (suggesting that they are processed from polycistronic primary transcripts), and that intronic miRNAs are usually coexpressed with their host gene mRNA (suggesting that they both derive from a common transcript) [15].

To examine this situation in ovarian cancer (thereby reinforcing current notions of miRNA biology as well as the integrity of the TCGA data), pairwise comparisons for each chromosome between the expression profiles of all miRNAs oriented in the same direction were made, calculating for each pair a correlation coefficient. The results showed that most miRNA genes within 50-100 kb of each other had highly correlated expression patterns. Notably, at distances beyond 100 kb (exceeding the length of most human genes), the correlation between pairs dropped dramatically to zero. While DNA copy number alterations undoubtedly influence gene and miRNA expression in cancer, pairwise correlations in copy number levels between proximal miRNAs showed a very different pattern from the pairwise expression correlations. High proximal correlations for copy number extended for >1 Mb in length, with no dramatic drop.

Approximately 177 of the 558 mature human miRNAs profiled are located in the genome within the introns of host genes, and miRNAs were found to be frequently coexpressed with these host genes in this data. For each of 188 miRNA-host gene pairs (each comprised of a miRNA located within the boundaries of a known gene, same orientation, where some mature miRNAs have multiple genomic locations), the correlation between miRNA and host gene expression was computed. miRNA-host gene pairs tended to be strongly correlated with each other and, with 52% of the miRNA-host gene pairs with available data showing significant positive correlation 1 (p<0.01), in agreement with previous studies. As expected, miRNA expression was also correlated with host gene copy number, though the correlations were not as strong as for gene expression.

### Example 4

MiRNAs and their Predicted Gene Targets Tend to be Anti-Correlated within Ovarian Tumors

A key to studying miRNAs is identifying their gene targets. While miRNA targeting predictions made in silico (the vast majority being unvalidated) may have sizable rates of false positives and negatives, considering correlations between gene and miRNA expression across a large panel of tumors could provide further support for potential miRNA:mRNA targeting relationships. To this end, all possible miRNA:mRNA correlations across the 487 TCGA ovarian tumors were computed, for the top expressed 191 miRNAs and 8547 genes. The 191×8547 miRNA:mRNA pairs were then sorted by low to high correlation, and found that among the most anti-correlated pairs, there was high enrichment for predicted miRNA:mRNA targeting interactions by miRanda algorithm, where no such enrichment was observed for the positively correlated miRNAs:mRNAs. (This trend was observed when considering all other miRNAs and genes in addition to those most highly expressed. In addition to validating the public target prediction databases as being enriched for true positives, this finding indicated that thousands of miRNA:mRNA targeting interactions are active in ovarian cancer and influence tumor gene expression heterogeneity.

The impact of copy number alteration on expression level can vary greatly between genes, conceivably introducing bias when evaluating association of miRNA and gene expression levels. Therefore, in addition to a direct Pearson's correlation between miRNA and mRNA, a simple linear regression model was applied to account for ‘noise’ due to copy number alteration, evaluating the association between expression levels of a miRNA and mRNA, when copy number alteration status of the gene is held fixed. Interestingly, the Pearson's model and the regression model of miRNA:mRNA correlations both gave very similar overall results in terms of predicted target enrichment, with the regression model's negatively correlated pairs showing slightly greater target enrichment. While, in general, copy number alteration did not represent a major confounding factor, the regression model could identify individual miRNA:mRNA correlations which were missed by the Pearson's model, including miR-29a:HARS2.

As another way to globally represent miRNA:mRNA interactions in ovarian cancer, for all miRNA:mRNA pairs with the strongest negative correlation (regression coefficient<−7.0, based on the linear model), the matrix of correlation coefficients were clustered, thereby grouping miRNAs when they are negatively correlated with same genes and vice versa. The gene dendrogram was then cut to extract 6 gene clusters (based on what appeared to be natural separations within the cluster tree), each of which was found to be uniquely enriched for different gene classes, including a cluster with Wnt and Hedgehog pathway gene members, a cluster with cell adhesion genes, two clusters with immune response genes, and a cluster of cell cycle-related genes. For several individual miRNAs, the genes anti-correlated in expression were significantly enriched for in silico predicted targets.

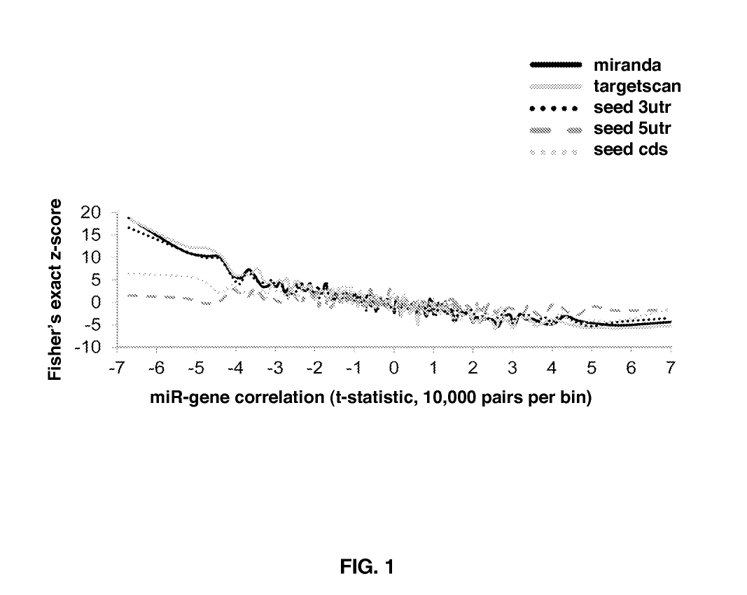

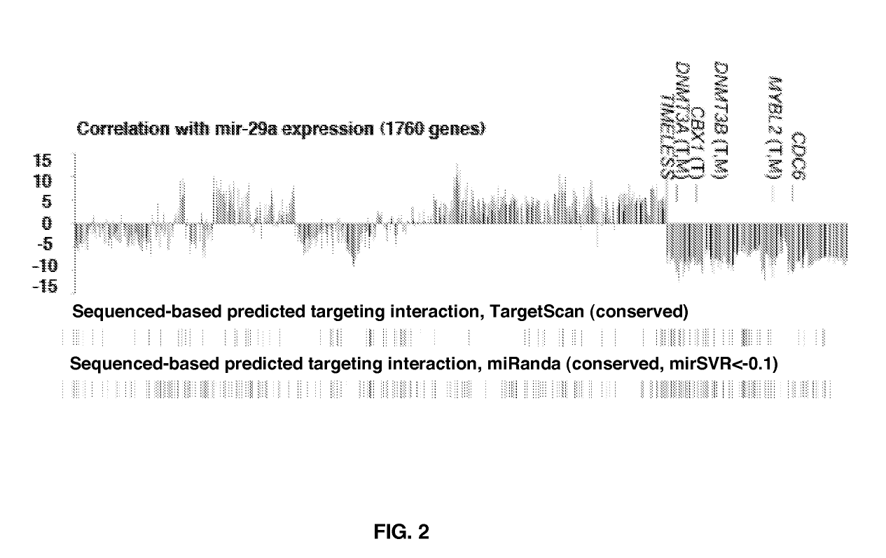

FIG. 1 shows that gene transcripts with miRNA 7mer in the 3′-UTR tend to be anti-correlated with expression of the corresponding miRNA. Top anticorrelated genes of miR-29 in ovarian cancer included DNMT3A and DNMT3B, suggesting a role for miR-29 in high-grade serous ovarian cancer. FIG. 2 shows the correlation of gene expression with miR-29a expression. MiR-29a was underexpressed in the DNA methylation subtype “MC2”. Genes anti-correlated with miR-29a were enriched for miR-29a targets as predicted by sequence analysis (either TargetScan or miRanda, FIG. 2). However, many in silico predicted targets did not show the anticipated anti-correlation patterns, again suggesting that by factoring in expression data, one could reduce the false positive rate for target predictions.

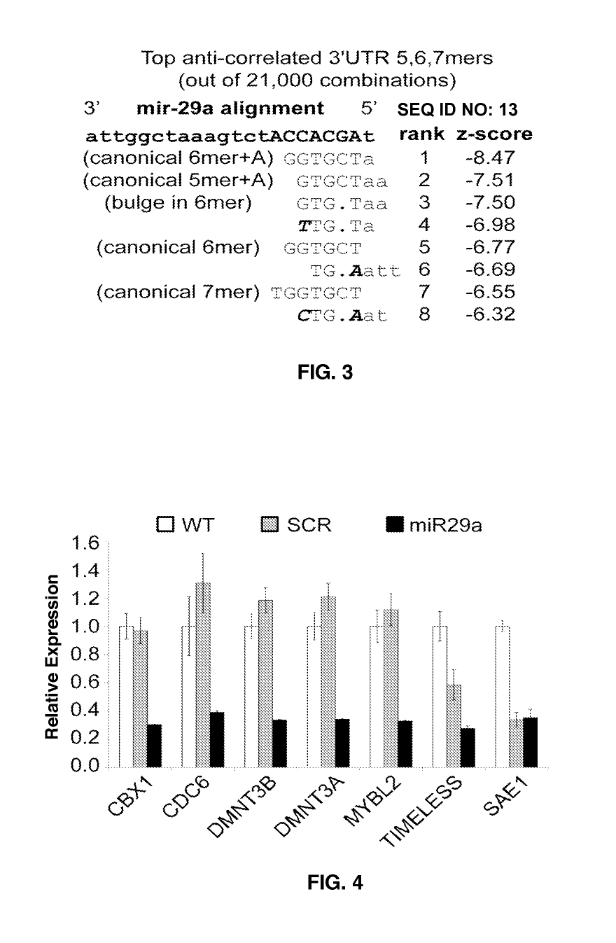

FIG. 3 shows the top eight words (of all 5, 6 and 7mers) enriched in 3′-UTRs of mRNAs anti-correlated with miR-29a (5′-TAGCACCATCTGAAATCGGTTA-3′, SEQ ID NO: 13) expression (FDR<1⁻⁶). FIG. 4 contains a QPCR analysis showing relative quantity of selected miR-29a anti-correlated gene targets after miR29a overexpression in HEYA8 ovarian cancer cells. Furthermore, as additional evidence for miR-29 activity, a correlation-based sequence motif analysis found that the miR-29 seed sequence complement was the top enriched motif in 3′-UTRs of mRNAs anti-correlated with miR-29a expression (FIG. 3), further suggesting that miR-29 directly regulates expression levels of many target mRNAs in the tumors. This analysis also showed strong enrichment for non-canonical miR-29a seed motifs (i.e. motifs not following the typical pattern of nucleotides 2-7) with a bulge in position 3 of the miR-29a sequence, suggesting that target prediction methods requiring perfect base pairing in the seed region of the miRNA target duplex could miss a substantial fraction of functional miRNA target interactions.

By forcing miR-29a expression in vitro in the ovarian cancer cell line HEYA8, it was confirmed that a number of the genes anti-correlated with miR-29a, i.e., DNMT3A, DNMT3B, CDC6, CBX1, MYBL2, and TIMELESS (four of which were predicted direct targets), were repressed by miR-29a (FIG. 4), which demonstrated these gene targets as relevant in both the in vitro functional models as well as the human tumor specimens; one gene tested, SAE1, showed anticorrelations but no functional repression.

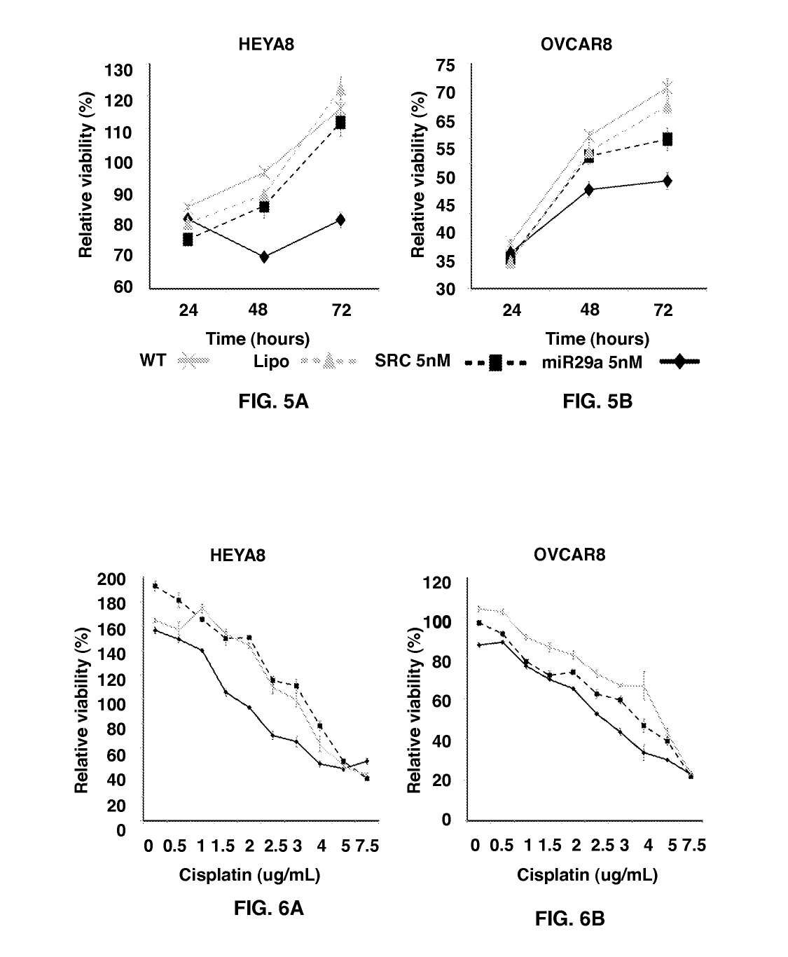

While miR-29 expression was not associated with survival (P>0.05, univariate Cox), forced miR-29a expression impacted cell proliferation in OvCar-8 and HEYA8 cell lines (FIGS. 5A-5B) and had an additional effect on chemotherapeutic agent cisplatin in inhibiting the growth of these lines (FIGS. 6A-6B). FIGS. 5A-5B demonstrate the effect of miR-29a overexpression on proliferation of HEYA8 and OVCAR-8 cells. When compared with the parental strains in each case as well as these cell lines transiently transfected with a scrambled control miR-29a is able to very significantly suppress cell proliferation in p53-wild type HEYA8 and moderately suppress cell proliferation of the p53-deficient OVCAR8. FIG. 6 shows the effect of miR-29a on proliferation under cisplatin treatment. As can be clearly seen by FIG. 6A, miR-29a suppresses the proliferation of HEYA8 significantly more effectively than the scrambled control at the same dosage of cisplatin.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. Also, the terms in the claims have their plain, ordinary meaning unless otherwise explicitly and clearly defined by the patentee.

What is claimed is:

1. A method of providing a prognosis for ovarian cancer in a subject, comprising the steps of:
obtaining a biological sample from said subject; and
testing said biological sample to determine whether or not microRNA 29 is under-expressed in said sample, relative to the expression of microRNA 29 in a control sample, whereby the under-expression of microRNA 29 in said biological sample indicates that a tumor in said subject is resistant to a chemotherapy.

2. A method of improving a therapeutic response to a cancer treatment, in a subject, the method comprising administering an effective amount of an agent that enhances the expression of microRNA 29 or an agent that mimics the effects of microRNA 29.

3. The method of claim 2, whereby said agent is microRNA 29a, microRNA 29b or microRNA 29c.

4. The method of claim 2, whereby said agent is a double-stranded miRNA mimic.

5. The method of claim 2, whereby said agent is an oligonucleotide based pre-microRNA 29 drug.

6. The method of claim 2, whereby said cancer is selected from the group consisting of lung cancer, pancreatic cancer, skin cancer, hematological neoplasms, breast cancer, brain cancer, colon cancer, follicular lymphoma, bladder cancer, cervical cancer, endometrial cancer, esophageal cancer, gastric cancer, head and neck cancer, multiple myeloma, liver cancer, lymphomas, oral cancer, osteosarcomas, ovarian cancer, uterine leiomyosarcoma, uterine leiomyomas, endometriomas, endometriosis, uterine papillary serous carcinomas, prostate cancer, testicular cancer and thyroid cancer.

7. The method of claim 6, whereby said cancer is epithelial ovarian cancer.

8. The method of claim 2, whereby said therapeutic response comprises treating with radiation, carboplatin, cisplatin, paclitaxel, an alkylating agent, an antimetabolite, an antitumor antibiotic and a DNA topoisomerase inhibitor.

9. A kit for determining a chemotherapy response in a patient with a cancer, said kit comprising: a) a oligonucleotide complementary to microRNA 29; and b) optionally, reagents for the formation of the hybridization between said oligonucleotide and said microRNA 29.

10. The kit according to claim 9, wherein said microRNA 29 is detectably labeled.

11. The kit according to claim 9, wherein said microRNA 29 is attached to a solid surface.

12. The kit according to claim 9, wherein said microRNA 29 is a member of a nucleic acid array.

13. The kit according to claim 12, wherein said nucleic acid array is a microarray.

14. A pharmaceutical composition for improving a tumor response to chemotherapy, said composition comprising an effective amount of microRNA 29 or an agent that enhances the expression of microRNA 29.

15. A method of treating a cancer in a subject in need of such treatment comprising the step of administering an effective amount of a microRNA 29 or an agent that enhances the expression of microRNA 29.

16. The method of claim 15, whereby said agent is microRNA 29a, microRNA 29b or microRNA 29c.

17. The method of claim 15, whereby said agent is a double-stranded miRNA mimic.

18. The method of claim 15, whereby said agent is an oligonucleotide based pre-microRNA 29 drug.

19. The method of claim 15, whereby said cancer is selected from the group consisting of lung cancer, pancreatic cancer, skin cancer, hematological neoplasms, breast cancer, brain cancer, colon cancer, follicular lymphoma, bladder cancer, cervical cancer, endometrial cancer, esophageal cancer, gastric cancer, head and neck cancer, multiple myeloma, liver cancer, lymphomas, oral cancer, osteosarcomas, ovarian cancer, uterine leiomyosarcoma, uterine leiomyomas, endometriomas, endometriosis, uterine papillary serous carcinomas, prostate cancer, testicular cancer and thyroid cancer.

20. The method of claim 19, whereby said cancer is epithelial ovarian cancer.

21. The method of claim 15, further comprising:
treating said subject with radiation, carboplatin, cisplatin, paclitaxel, an alkylating agent, an antimetabolite, an antitumor antibiotic and a DNA topoisomerase inhibitor.

22. The method of claim 15, wherein said microRNA 29 is administered as a nucleic acid construct encoding an artificial miRNA presented as a double-stranded RNA, a precursor hairpin, a primary miRNA in single straded RNA form or encoded in a DNA vector delivered in a suitable pharmaceutical carrier.

23. The method of claim 22, wherein said pharmaceutical carrier is selected from the group consisting of a virus, a liposome, and a polymer.

24. The method of claim 15, wherein said microRNA 29 is administered as a nanoparticle, a liposome, a vector or a polymer.

25. The method of claim 24, wherein said vector selected from the group consisting of a plasmid, a cosmid, a phagemid and a virus.
